(19) United States
(12) Patent Application Publication
(10) Pub. No.: US 2026/0184653 A1
(43) Pub. Date: Jul. 2, 2026

(54) METHODS OR DEHYDROGENATING HYDROCARBONS UTILIZING MULTIPLE CATALYST INLETS

(71) Applicant: Dow Global Technologies LLC, Midland, MI (US)

(72) Inventors: Lin Luo, Sugarland, TX (US); Hangyao Wang, Pearland, TX (US); Matthew T. Pretz, Lake Jackson, TX (US); Quan Yuan, Sugarland, TX (US); Liwei Li, Houston, TX (US); Jeffry A. Ferrio, Midland, MI (US)

(73) Assignee: Dow Global Technologies LLC, Midland, MI (US)

(21) Appl. No.: 19/131,637

(22) PCT Filed: Nov. 27, 2023
(86) PCT No.: PCT/US2023/081074

§ 371 Date: May 21, 2025

Related U.S. Application Data
(60) Provisional application No. 63/428,521, filed on Nov. 29, 2022.

(51) Int. Cl. C07C 5/32

(52) U.S. Cl. CPC C07C 5/325; C07C 2523/62

(57) ABSTRACT

A method for producing one or more olefinic compounds may include dehydrogenating a feed stream in the presence of a catalyst in a reactor to form a product stream and a deactivated catalyst. The feed stream may include one or more hydrocarbons comprising an alkyl moiety. The product stream may include one or more olefinic compounds. The method may also include separating the deactivated catalyst into a first portion of deactivated catalyst and a second portion of deactivated catalyst. The method may include passing the second portion of deactivated catalyst to a regenerator. The method may include processing the second portion of deactivated catalyst in the regenerator to form a regenerated catalyst. The method may also include passing the first portion of deactivated catalyst and the regenerated catalyst to the reactor. The first portion of deactivated catalyst may enter the reactor downstream of the regenerated catalyst relative to the flow direction of the feed stream. The first portion of deactivated catalyst may have a lower temperature than the regenerated catalyst.

### CROSS-REFERENCE TO RELATED APPLICATIONS

This application claims the benefit of U.S. Provisional Application Ser. No. 63/428,521 filed Nov. 29, 2022, the entire disclosure of which is hereby incorporated herein by reference.

### TECHNICAL FIELD

Embodiments described herein generally relate to chemical processing and, more specifically, to processes and systems utilized for the dehydrogenation of chemical species.

### BACKGROUND

Olefinic compounds may be utilized as base materials to produce many types of goods and materials. For example, propylene may be utilized to manufacture polypropylene, propylene oxide, and acrylonitrile. Such products may be utilized in product packaging, chemical manufacturing, textiles, etc. Thus, there is an industry demand for olefinic compounds, such as ethylene, propylene, butene, and styrene, as well as processes to produce such materials.

### SUMMARY

One method for producing olefinic compounds is by dehydrogenating hydrocarbons. In some embodiments, the dehydrogenation reaction may be promoted by utilizing a solid particulate catalyst in a circulating fluidized bed (CFB) system. In embodiments, the catalyst may become deactivated as it is utilized in the dehydrogenation reaction. Such deactivated catalyst may be passed to a regenerator to restore at least a portion of the catalyst's activity, such as by decoking the catalyst or heating the catalyst. Alternatively, some of the deactivated catalyst may be recycled and reused in the dehydrogenation reaction without being regenerated.

As is described herein, it has been discovered that it may be beneficial to utilize particular catalyst distribution patterns with respect to the recycled reaction catalyst, regenerated catalyst, and the feed stream entering the reactor. Embodiments described herein include catalyst distribution patterns where the recycled reaction catalyst may enter the reactor downstream of the regenerated catalyst relative to the flow direction of the feed stream. Such catalyst distribution patterns may, for example, improve catalyst activity in the reactor and improve yield from the dehydrogenation reaction when compared to catalyst distribution patterns that do not have the recycled reaction catalyst enter the reactor downstream of the regenerated catalyst relative to the flow direction of the feed stream. Generally, the recycled reaction catalyst is cooler than the regenerated catalyst and has less catalytic activity. One skilled in the art would expect premixing of the two streams of catalyst would help reduce spatial variation of catalyst activity and temperature in the reactor and be advantageous to the process. However, contrary to what would be expected by one skilled in the art, the presently described methods unexpectedly offer superior yield when compared to embodiments where the recycled reaction catalyst premixes with the regenerated catalyst by having the less catalytically active recycled reaction catalyst contact the feed only after contacting the more catalytically active regenerated catalyst with the feed.

According to one or more embodiments described herein, a method for producing one or more olefinic compounds may include dehydrogenating a feed stream in the presence of a catalyst in a reactor to form a product stream and a deactivated catalyst. The feed stream may comprise one or more hydrocarbons comprising an alkyl moiety. The product stream may comprise one or more olefinic compounds. The method may also include separating the deactivated catalyst into a first portion of deactivated catalyst and a second portion of deactivated catalyst. The method may include passing the second portion of deactivated catalyst to a regenerator. The method may include processing the second portion of deactivated catalyst in the regenerator to form a regenerated catalyst. The method may also include passing the first portion of deactivated catalyst and the regenerated catalyst to the reactor. The first portion of deactivated catalyst may enter the reactor downstream of the regenerated catalyst relative to the flow direction of the feed stream. The first portion of deactivated catalyst may have a lower temperature than the regenerated catalyst.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawings and claims, or recognized by practicing the described embodiments. The drawings are included to provide a further understanding of the embodiments and, together with the detailed description, serves to explain the principles and operations of the claimed subject matter. However, the embodiments depicted in the drawings are illustrative and exemplary in nature, and not intended to limit the claimed subject matter.

### BRIEF DESCRIPTION OF THE DRAWINGS

Reference will now be made in greater detail to various embodiments, some of which are illustrated in the accompanying drawings, wherein:

FIG. 1 schematically depicts a reactor system, according to one or more embodiments of the present disclosure;

FIG. 2 schematically depicts a reactor, according to one or more embodiments of the present disclosure;

FIG. 3A schematically depicts a continuous stir tank reactor system configuration;

FIG. 3B schematically depicts a continuous stir tank reactor system configuration; and

FIG. 3C schematically depicts a continuous stir tank reactor system configuration.

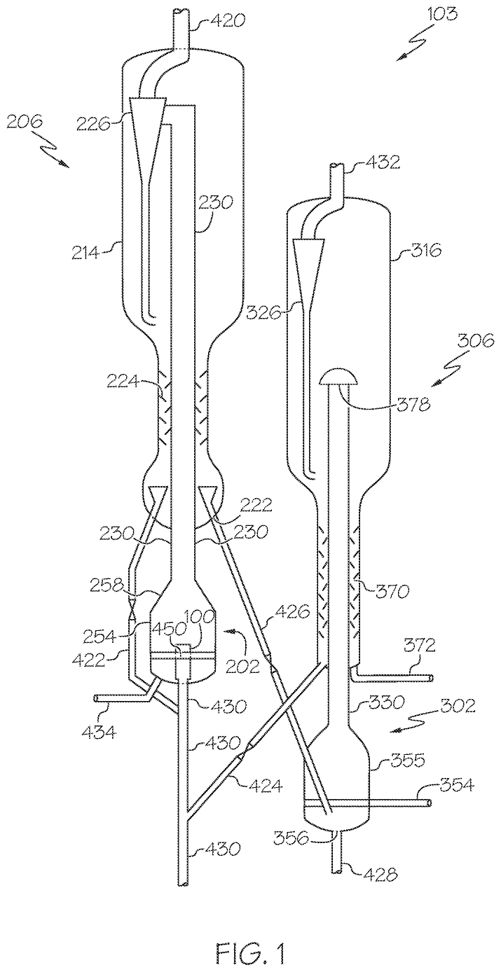

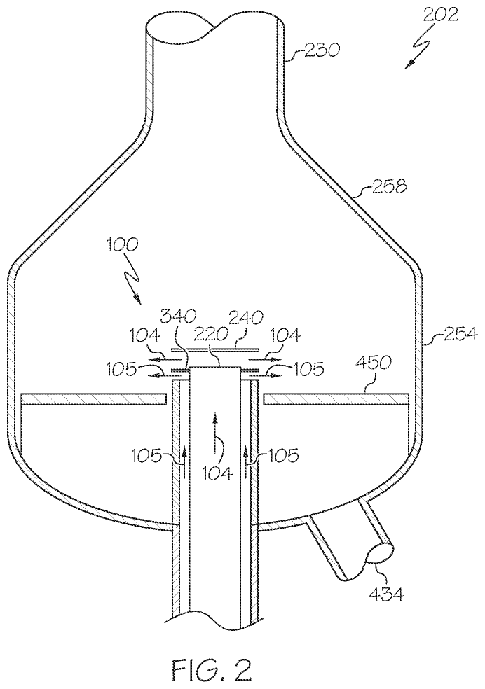

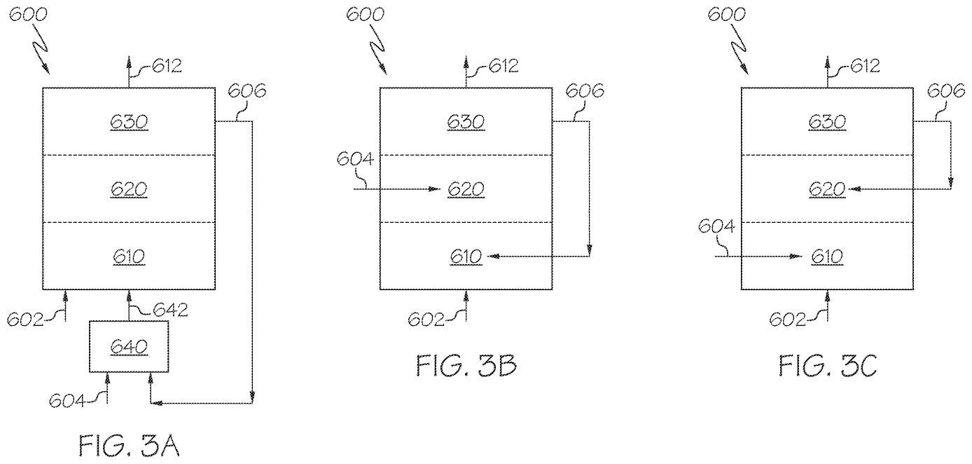

When describing the simplified schematic illustration of FIG. 1 FIG. 2, FIG. 3A, FIG. 3B, and FIG. 3C, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

### DETAILED DESCRIPTION

Specific embodiments of the present application will now be described. The disclosure may, however, be embodied in different forms and should not be construed as limited to the embodiments set forth in this disclosure. Rather, these embodiments are provided so that this disclosure will be thorough and complete, and will fully convey the scope of the subject matter to those skilled in the art.

Embodiments presently disclosed will now be described in detail herein in the context of the reactor system of FIG. 1 operating as a fluidized dehydrogenation reactor system to produce olefinic compounds, such as propylene. However, it should be understood that the principles disclosed and taught herein may be applicable to other systems which utilize different system components oriented in different ways, or different reaction schemes utilizing various catalyst compositions. For example, the concepts described may be equally applied to other systems with alternate reactor units and regeneration units, such as those that operate under non-fluidized conditions or include downers rather than risers. It should be further understood that not all portions of the reactor system of FIG. 1 should be construed as essential to the claimed subject matter.

Now referring to FIG. 1, an example reactor system 103 that may be suitable for use with the methods and/or apparatuses described herein is schematically depicted. The reactor system 103 generally comprises multiple system components, such as a reactor portion 206 and a regeneration unit 306. As described herein, “system components” refer to portions of the reactor system 103, such as reactors, separators, transfer lines, combinations thereof, and the like. As used herein in the context of FIG. 1, the reactor portion 206 generally refers to the portion of a reactor system 103 in which the major process reaction takes place (e.g., dehydrogenation) to form the product stream. A feed stream enters the reactor portion 206 via feed inlet 434, is converted to a product stream (containing product and unreacted feed), and exits the reactor portion 206. The reactor portion 206 comprises a reactor 202, which may include an upstream reactor section 254 and a downstream reactor section 230. According to one or more embodiments, as depicted in FIG. 1, the reactor portion 206 may include a catalyst separation section 214, which serves to separate the catalyst from the chemical products formed in the reactor 202. The catalyst leaving the reactor 202 and passed to the catalyst separation section 214 may be deactivated and may, for example, be cooler or less active than the catalyst passed to the reactor 202 from the regeneration unit 306 via line 424.

In one or more embodiments, the catalyst may be separated into multiple portions of catalyst, exiting via lines 422 and 426. As described herein, a “first portion” of deactivated catalyst is passed via line 422 back to the reactor 202, in a recycle stream (not going to the combustor 355). A “second portion” of deactivated catalyst is passed via line 426 to the regeneration unit 306. As described herein, the first portion of catalyst and a “regenerated catalyst” (i.e., the regenerated version of the second portion of catalyst passed via line 424 to the reactor 202 are generally introduced into the reactor 202 separately and at different portions, such as different heights, of the reactor 202. Such a configuration may have a positive effect on catalytic efficiency.

As used, herein the regeneration unit 306 generally refers to the portion of the reactor system 103 where the catalyst is in some way processed, such as by combustion, to, e.g., improve catalytic activity and/or heat the catalyst. The regeneration unit 306 may comprise a combustor 355 and a riser 330, a particulate solid separation section 316, and may additionally comprise an oxygen treatment zone 370. In one or more embodiments, the particulate solid separation section 214 may be in fluid communication with the combustor 355 (e.g., via standpipe 426) and the particulate solid separation section 316 may be in fluid communication with the upstream reactor section 250 (e.g., via standpipe 424 and transport riser 430).

Generally, as is described herein, in embodiments illustrated in FIG. 1, the catalyst is cycled between the reactor portion 200 and the regeneration unit 300. It should be understood that when catalysts are referred to herein, they may refer to solid materials that are catalytically active for a desired reaction, or may equally refer to other particulate solids referenced with respect to the system of FIG. 1 which do not necessarily have catalytic activity but affect the reaction. The terms “catalytic activity” and “catalyst activity” refer to the degree to which the catalyst is able to catalyze the reactions conducted in the reactor system 106. In embodiments, the second portion of deactivated catalyst may be reactivated by catalyst reactivation in the regeneration unit 306. Reactivation (sometimes called “regeneration” herein) may remove contaminants such as coke, raise the temperature of the particulate solid, or both. The deactivated catalyst may be reactivated by, but not limited to, removing coke by combustion, recovering catalyst acidity, oxidizing the particulate solid, other reactivation process, or combinations thereof. The regenerated catalyst from the regeneration unit 306 may then be passed back to the reactor portion 202 via line 424.

Now referring to FIG. 1 in detail, the reactor portion 206 may comprise an upstream reactor section 254, a transition section 258, and a downstream reactor section 230, such as a riser. The transition section 258 may connect the upstream reactor section 254 with the downstream reactor section 230. As depicted in FIG. 1, the upstream reactor section 254 may be positioned below the downstream reactor section 230. Such a configuration may be referred to as an upflow configuration in the reactor 202. The upstream reactor section 254 may include a vessel, drum, barrel, vat, or other container suitable for a given chemical reaction. As depicted in FIG. 1, the upstream reactor section 254 may be connected to the downstream reactor section 230 via the transition section 258. The upstream reactor section 254 may generally comprise a greater cross-sectional area than the downstream reactor section 230. The transition section 258 may be tapered from the size of the cross-section of the upstream reactor section 254 to the size of the cross-section of the downstream reactor section 230 such that the transition section 258 projects inwardly from the upstream reactor section 254 to the downstream reactor section 230. For example, the transition section 258 may be a frustum.

As described with respect to FIG. 1, the feed stream may enter into the reactor 202 via feed inlet 434, and the product stream may exit the reactor system 103 via pipe 420. According to one or more embodiments, the reactor system 103 may be operated by feeding a chemical feed (e.g., in a feed stream) and a fluidized catalyst into the upstream reactor section 254. The chemical feed contacts the catalyst in the upstream reactor section 254, and each flow upwardly into and through the downstream reactor section 230 to produce a chemical product.

The upstream reactor section 254 may be connected to a transport riser 430, which, in operation may provide regenerated catalyst in a feed stream to the reactor portion 206. In one or more embodiments, the first portion of deactivated catalyst may enter the reactor downstream of the regenerated catalyst relative to the flow direction of the feed stream. As will be described herein, in one or more embodiments, the regenerated catalyst and the first portion of the deactivated catalyst may enter the reactor 202 via a particulate solids distributor 100. The particulate solids distributor 100 may separately pass the regenerated catalyst and the first portion of deactivated catalyst into the reactor 202. The catalyst entering the upstream reactor section 254 via transport riser 430 may be passed through line 424 to a transport riser 430, thus arriving from the regeneration unit 306. The first portion of deactivated catalyst may come directly from the catalyst separation section 214 via line 422 and into the transport riser 430, where it enters the upstream reactor section 254. This catalyst may be somewhat deactivated, but may still, in some embodiments, be suitable for reaction in the upstream reactor section 254, particularly when used in combination with the regenerated catalyst. The regenerated catalyst arriving from the regeneration unit 306 and the first portion of deactivated catalyst arriving from catalyst separation section 214 via line 422 may be kept separate within the transport riser 430 before being passed into the reactor 202 via particulate solids distributor 100.

Still referring to FIG. 1, in one or more embodiments, based on the shape, size, and other processing conditions (such as temperature and pressure) in the upstream reactor section 254 and the downstream reactor section 230, the upstream reactor section 254 may operate as a fluidized bed, such as in a fast fluidized, turbulent, or bubbling bed upflow reactor, while the downstream reactor section 230 may operate in more of a plug flow manner, such as in a riser reactor. For example, the reactor 202 of FIG. 1 may comprise an upstream reactor section 254 operating as a fast fluidized, turbulent, or bubbling bed reactor and a downstream reactor section 230 operating as a dilute phase riser reactor, with the result that the average catalyst and gas flow moves concurrently upward. As the term is used herein, “average flow” refers to the net flow, i.e., the total upward flow minus the retrograde or reverse flow, as is typical of the behavior of fluidized particles in general. As described herein, a “fast fluidized” reactor may refer to a reactor utilizing a fluidization regime wherein the superficial velocity of the gas phase is greater than the choking velocity and may be semi-dense in operation. As described herein, a “turbulent” reactor may refer to a fluidization regime where the superficial velocity of less than the choking velocity and is more dense than the fast fluidized regime. As described herein, a “bubbling bed” reactor may refer to a fluidization regime wherein well defined bubbles in a highly dense bed are present in two distinct phases. The “choking velocity” refers to the minimum velocity required to maintain solids in the dilute-phase mode in a vertical conveying line. As described herein, a “dilute phase riser” may refer to a riser reactor operating at transport velocity, where the gas and catalyst have about the same velocity in a dilute phase.

According to embodiments, the chemical product and the catalyst may be passed out of the downstream reactor section 230 to a separation device 226 in the catalyst separation section 214, where the catalyst is separated from the chemical product, which is transported out of the catalyst separation section 214. According to one or more embodiments, following separation from vapors in the separation device 226, the deactivated catalyst may generally move through the strip zone 224, where the first portion of deactivated catalyst is passed out of the strip zone 224 to the reactor 202 via line 422 and the second portion of deactivated catalyst passes to the catalyst outlet port 222 where the second portion of deactivated catalyst is transferred out of the reactor portion 206 via line 426 and into the regeneration unit 306.

Now referring back to FIG. 1, according to one or more embodiments, the separation device 226 may be a cyclonic separation system, which may include two or more stages of cyclonic separation. In embodiments where the separation device 226 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the catalyst from the product gas. It should be understood that any primary cyclonic separation device may be used in embodiments of the invention.

As described previously herein, the deactivated catalyst in the catalyst separation section 214 may be separated into a first portion of deactivated catalyst passed via line 422 back to the reactor 202 and a second portion of deactivated catalyst passed via line 426 to the combustor 350. The mass flow rate ratio of the first portion of deactivated catalyst to the second portion of deactivated catalyst may be from 0.1 to 5 For example, the mass flow rate ratio of the first portion of deactivated catalyst to the second portion of deactivated catalyst may be from 0.1 to 4.5, such as from 0.1 to 4, from 0.1 to 3.5, from 0.1 to 3, from 0.1 to 2.5, from 0.1 to 2, from 0.1 to 1.5, from 0.1 to 1, from 0.1 to 0.5, from 0.5 to 5, from 0.5 to 4.5, from 0.5 to 4, from 0.5 to 3.5, from 0.5 to 3, from 0.5 to 2.5, from 0.5 to 2, from 0.5 to 1.5, from 0.5 to 1, from 1 to 5, from 1 to 4.5, from 1 to 4, from 1 to 3.5, from 1 to 3, from 1 to 2.5, from 1 to 2, from 1 to 1.5, from 1.5 to 5, from 1.5 to 4.5, from 1.5 to 4, from 1.5 to 3.5, from 1.5 to 3, from 1.5 to 2.5, from 1.5 to 2, from 2 to 5, from 2 to 4.5, from 2 to 4, from 2 to 3.5, from 2 to 3, from 2 to 2.5, from 2.5 to 5, from 2.5 to 4.5, from 2.5 to 4, from 2.5 to 3.5, from 2.5 to 3, from 3 to 5, from 3 to 4.5, from 3 to 4, from 3 to 3.5, from 3.5 to 5, from 3.5 to 4.5, from 3.5 to 4, from 4 to 5, from 4 to 4.5, from 4.5 to 5, or any combination of these ranges. The mass flow rate of second portion of deactivated catalyst, which is passed to the combustor 350, may be about the same as the mass flow rate of regenerated catalyst passed to the reactor 202 via line 424, as is later discussed herein.

In one or more embodiments, the first portion of deactivated catalyst (passed via line 422) may have a temperature that is from 580° C. to 800° C., such as from 580° C. to 775° C., from 580° C. to 750° C., from 580° C. to 725° C., from 580° C. to 700° C., from 580° C. to 675° C., from 580° C. to 650° C., from 580° C. to 625° C., from 580° C. to 600° C., from 600° C. to 800° C., from 600° C. to 775° C., from 600° C. to 750° C., from 600° C. to 725° C., from 600° C. to 700° C., from 600° C. to 675° C., from 600° C. to 650° C., from 600° C. to 625° C., from 625° C. to 800° C., from 625° C. to 775° C., from 625° C. to 750° C., from 625° C. to 725° C., from 625° C. to 700° C., from 625° C. to 675° C., from 625° C. to 650° C., from 650° C. to 800° C., from 650° C. to 775° C., from 650° C. to 750° C., from 650° C. to 725° C., from 650° C. to 700° C., from 650° C. to 675° C., from 675° C. to 800° C., from 675° C. to 775° C., from 675° C. to 750° C., from 675° C. to 725° C., from 675° C. to 700° C., from 700° C. to 800° C., from 700° C. to 775° C., from 700° C. to 750° C., from 700° C. to 725° C., from 725° C. to 800° C., from 725° C. to 775° C., from 725° C. to 750° C., from 750° C. to 800° C., from 750° C. to 775° C., from 775° C. to 800° C., or any combination of these ranges. This is generally less than the temperature of the regenerated catalyst passed to the reactor 202 via line 424.

Still referring to FIG. 1, the second portion of deactivated catalyst may be passed via line 426 from the catalyst separation section 214 to the combustor 355. In the combustor 355, the second portion of deactivated catalyst may be processed by, for example, combustion of any coke on the catalyst with oxygen or with a supplemental fuel. For example, and without limitation, the catalyst may be de-coked and/or fuel may be combusted to heat the catalyst. The catalyst is then passed out of the combustor 355 and through the riser 330 to a riser termination separator 378, where the gas and solid components from the riser 330 are at least partially separated. The vapor and remaining solids are transported to a secondary separation device 326 in the catalyst separation section 316 where the remaining catalyst is separated from the gases from the catalyst processing (e.g., gases emitted by combustion of spent catalyst or fuel, referred to herein as flue gas). The flue gas may pass out of the regeneration unit 306 via outlet pipe 432. The separated catalyst is then passed through the oxygen treatment zone 370 within the catalyst separation section 316 to the upstream reactor section 254 via line 424 and transport riser 430, where it is further utilized in a catalytic reaction. This catalyst, processed as described, is referred to as a “regenerated catalyst” and is passed back to the reactor 202 in a separate stream from the first portion of deactivated catalyst.

In one or more embodiments, the regenerated catalyst may have a temperature of from 680° C. to 900° C. when it is passed to the reactor 202 from the regeneration unit 306 via line 424. For example, the regenerated catalyst may have a temperature of from 680° C. to 875° C., such as from 680° C. to 850° C., from 680° C. to 825° C., from 680° C. to 800° C., from 680° C. to 775° C., from 680° C. to 750° C., from 680° C. to 725° C., from 680° C. to 700° C., from 700° C. to 900° C., from 700° C. to 875° C., from 700° C. to 850° C., from 700° C. to 825° C., from 700° C. to 800° C., from 700° C. to 775° C., from 700° C. to 750° C., from 700° C. to 725° C., from 725° C. to 900° C., from 725° C. to 875° C., from 725° C. to 850° C., from 725° C. to 825° C., from 725° C. to 800° C., from 725° C. to 775° C., from 725° C. to 750° C., from 750° C. to 900° C., from 750° C. to 875° C., from 750° C. to 850° C., from 750° C. to 825° C., from 750° C. to 800° C., from 750° C. to 775° C., from 775° C. to 900° C., from 775° C. to 875° C., from 775° C. to 850° C., from 775° C. to 825° C., from 775° C. to 800° C., from 800° C. to 900° C., from 800° C. to 875° C., from 800° C. to 850° C., from 800° C. to 825° C., from 825° C. to 900° C., from 825° C. to 875° C., from 825° C. to 850° C., from 850° C. to 900° C., from 850° C. to 875° C., from 875° C. to 900° C., or any combination of these ranges.

Referring now to the regeneration unit 306, as depicted in FIG. 1, the combustor 355 of the regeneration unit 306 may include one or more lower combustor inlet ports 356 and may be in fluid communication with the riser 330. Oxygen-containing gas, such as air, may be passed through pipe 428 into the combustor 350. The combustor 355 may be in fluid communication with the catalyst separation section 214 via line 426, which may supply the second portion of deactivated catalyst from the reactor portion 206 to the regeneration unit 306 for regeneration. The combustor 355 and riser 330, collectively referred to as the catalyst combustion reactor 302, may operate with similar or identical fluidization regimes as to what was disclosed with respect to the upstream reactor section 254 and downstream reactor section 230 of the reactor portion 206. That is, the combustor 350 may operate as a fluidized bed, such as in a fast fluidized, turbulent, or bubbling bed upflow reactor, while the riser 330 may operate in more of a plug flow manner, such as in a riser reactor. Geometries as described with respect to the upstream reactor section 254 and downstream reactor section 230 may equally apply to the combustor 355 and riser 330. Additionally, the combustor 355 may also include a fuel inlet 354, which may supply a fuel, such as a hydrocarbon stream or hydrogen, to the combustor 355.

As described in one or more embodiments, following separation of flue gas from catalyst in the riser termination separator 378 and secondary separation device 326, treatment of the processed catalyst with an oxygen-containing gas is conducted in the oxygen treatment zone 370. In some embodiments, the oxygen treatment zone 370 includes a fluid solids contacting device. The fluid solids contacting device may include baffles or grid structures to facilitate contact of the processed catalyst with the oxygen-containing gas. Examples of fluid solid contacting devices are described in further detail in U.S. Pat. Nos. 9,827,543 and 9,815,040. The fluidization regime within the oxygen treatment zone 370 may be bubbling bed type fluidization.

As described herein, the regenerated catalyst and first portion of deactivated catalyst enter the reactor 202 in separate streams. In one or more embodiments, the first portion of deactivated catalyst and the regenerated catalyst may make up at least 95 wt. % of the catalyst passed to the reactor 202. For example, the first portion of deactivated catalyst and the regenerated catalyst may make up at least 95 wt. %, at least 96 wt. %, at least 97 wt. %, at least 98 wt. %, at least 99 wt. %, or even at least 99.9 wt. % of the catalyst passed to the reactor 202.

As is described in one particular embodiment with respect to FIG. 2 herein below, in one or more embodiments, the first portion of deactivated catalyst and the regenerated catalyst may enter the reactor 202 through separate distributors. In one or more embodiments, the first portion of deactivated catalyst and the regenerated catalyst may not enter the reactor 202 through the bottom end of the reactor 202.

Now referring to FIG. 2, an example reactor 202 that may be utilized with the reactor system 103 of FIG. 1, is schematically depicted, which includes a particulate solids distributor 100 suitable to pass the regenerated catalyst and first portion of deactivated catalyst at different heights. FIG. 2 depicts one contemplated particulate solids distributor. However, other solids distributors may be suitable, and the embodiments described herein should not be construed as limited by the design, shape, size, architecture, etc. of the distributor or distributors that pass particulate solids into the combustor 350.

Now referring to FIG. 2, the reactor 202 of the reactor system 103 of FIG. 1 is schematically depicted. The first portion of deactivated catalyst 104 and the regenerated catalyst 105 may be passed into the reactor 202 through the particulate solids distributor 100. The particulate solids distributor 100 may pass the first portion of deactivated catalyst 104 into the reactor 202 downstream of the regenerated catalyst 105 relative to the flow direction of the feed stream, which enters the reactor 202 through feed inlet 434 and through feed distribution plate 450. The first portion of deactivated catalyst 104 may be passed up an inner section of the particulate solids distributor 100 to an inner conduit outlet 220. The first portion of deactivated particulate solid 104 may then contact a first catalyst director 240 which may direct the first portion of deactivated catalyst 104 into the reactor 202. The regenerated catalyst 104 may pass through an outer section of the particulate solids distributor to contact a second catalyst director 340 that may direct the regenerated catalyst 105 into the reactor 202. As shown in FIG. 2, in one or more embodiments, the regenerated catalyst 105 may enter the reactor 202 between the feed stream and the first portion of deactivated catalyst 104. As the feed stream passes from feed inlet 434 into the reactor 202 it passes in an upward direction from upstream reactor section 254 to downstream reactor section 230. As the feed stream travels through the reactor 202 it first contacts the regenerated catalyst 105 being directed into the reactor 202 by the second catalyst director 340. At least a portion of the feed stream may react with the regenerated catalyst 105 producing one or more products. A mixed stream which may comprise one or more products, the regenerated catalyst 105, and the feed stream continues upward through the reactor to contact the first portion of deactivated catalyst 104 which is directed into the reactor 202 by the first catalyst director 240. The first portion of deactivated catalyst 104 may contact the mixed stream and may react with unreacted feed from the feed stream to produce one or more products. The mixture of the regenerated catalyst 105, the first portion of deactivated catalyst 104, the one or more products, and any remaining unreacted feed travels to the upstream reactor section 230, where it may be passed to the catalyst separation section 214 of the reactor system 103 of FIG. 1.

Without being bound by any particular theory, and with reference to the examples that follow, it is unexpectedly found that the catalyst input patterns described herein may, in some embodiments, produce superior outcomes as compared with embodiments that do not include the described catalyst input patterns. In particular, it would have been expected that introducing lower temperature recycled catalyst upstream of the higher temperature regenerated catalyst would deliver better selectivity and subsequently higher yield. It would have also been expected that premixing of the two streams of catalyst would help reduce spatial variation of catalyst activity and temperature in the reactor and be advantageous for better yield. However, as is demonstrated in the present Examples, unexpectedly, the opposite is the case.

In one or more additional embodiments, hydrocarbons, such as for example, methane and ethane, may be entrained in the first portion of deactivated catalyst. In one or more embodiments, hydrocarbons may be entrained in the first portion of deactivated catalyst when it is passed back to the reactor 202 via line 422. In the regeneration unit the entrained hydrocarbon in the second portion of deactivated catalyst may be combusted, and the regenerated catalyst passed back to the reactor 202 from the regeneration unit 306 via line 424 may have no or substantially no entrained hydrocarbons, such as less than 0.05 mol % of entrained hydrocarbons in the regenerated catalyst. However, there could be oxygen-containing gas entrained by the regenerated catalyst particles. Without intending to be bound by theory, it is believed that when the hydrocarbons entrained in the first portion of deactivated catalyst contact the oxygen entrained regenerated catalyst with high temperatures (such as in comparative embodiments), coke and steam may form on the regenerated catalyst. This may partially deactivate at least a portion of the regenerated catalyst. For example, contacting the high-temperature regenerated catalyst with hydrocarbons may partially deactivate the regenerated catalyst before the catalyst has an opportunity to contact the feed stream in the reactor, which may reduce the efficiency of the reactor system 103. It is believed that introducing the first portion of deactivated catalyst and the regenerated catalyst into the reactor at different positions may prevent or reduce premature deactivation of the regenerated catalyst, as the entrained hydrocarbons in the first portion of deactivated catalyst may not contact the regenerated catalyst prior to the regenerated catalyst entering the reactor 202. Further, as the first portion of deactivated catalyst may be passed into the reactor 202 downstream of the regenerated catalyst relative to the flow direction of the feed stream, the regenerated catalyst may contact the feed stream before contacting the first portion of deactivated catalyst and any entrained hydrocarbons in the first portion of deactivated catalyst.

As was described with respect to FIG. 2, in one or more embodiments, the reactor 202 may comprise a feed distribution plate 450, as shown in FIG. 1 and FIG. 2. In one or more embodiments, where the reactor 202 comprises a feed distribution plate 450 the regenerated catalyst may enter the reactor 202 between the feed distribution plate 450 and the first portion of deactivated catalyst.

In one or more embodiments, the temperature of the feed distribution plate may be from 25° C. to 700° C. For example, the temperature of the feed distribution plate may be from 25° C. to 600° C., from 25° C. to 500° C., from 25° C. to 400° C., from 25° C. to 300° C., from 25° C. to 200° C., from 25° C. to 100° C., from 100° C. to 700° C., from 100° C. to 600° C., from 100° C. to 500° C., from 100° C. to 400° C., from 100° C. to 300° C., from 100° C. to 200° C., from 200° C. to 700° C., from 200° C. to 600° C., from 200° C. to 500° C., from 200° C. to 400° C., from 200° C. to 300° C., from 300° C. to 700° C., from 300° C. to 600° C., from 300° C. to 500° C., from 300° C. to 400° C., from 400° C. to 700° C., from 400° C. to 600° C., from 400° C. to 500° C., from 500° C. to 700° C., from 500° C. to 600° C., from 600° C. to 700° C., or any combination of these ranges.

In one or more embodiments, the deactivated catalyst may be separated in the catalyst separation section 214 into a third portion of deactivated catalyst (not depicted in FIG. 1). In such embodiments, the regenerated catalyst may be mixed with the third portion of deactivated catalyst prior to being introduced into the reactor 202 to form a mixed catalyst which is then passed into the reactor 202. In such embodiments, the first portion of deactivated catalyst may be passed into the reactor 202 downstream of the mixed catalyst relative to the flow direction of the feed stream. Without intending to be bound by theory, it is believed that mixing regenerated catalyst with the third portion of deactivated catalyst to form the mixed catalyst may have less negative impact on the activity of regenerated catalyst due to a lower amount of hydrocarbons entrained in the third portion of deactivated catalyst.

In one or more embodiments, the mixed catalyst may have a temperature that is from 600° C. to 850° C. For example, the mixed catalyst may have a temperature that is from 600° C. to 825° C., from 600° C. to 800° C., from 600° C. to 775° C., from 600° C. to 750° C., from 600° C. to 725° C., from 600° C. to 700° C., from 600° C. to 675° C., from 600° C. to 650° C., from 600° C. to 625° C., from 625° C. to 850° C., from 625° C. to 825° C., from 625° C. to 800° C., from 625° C. to 775° C., from 625° C. to 750° C., from 625° C. to 725° C., from 625° C. to 700° C., from 625° C. to 675° C., from 625° C. to 650° C., from 650° C. to 850° C., from 650° C. to 825° C., from 650° C. to 800° C., from 650° C. to 775° C., from 650° C. to 750° C., from 650° C. to 725° C., from 650° C. to 700° C., from 650° C. to 675° C., from 675° C. to 850° C., from 675° C. to 825° C., from 675° C. to 800° C., from 675° C. to 775° C., from 675° C. to 750° C., from 675° C. to 725° C., from 675° C. to 700° C., from 700° C. to 850° C., from 700° C. to 825° C., from 700° C. to 800° C., from 700° C. to 775° C., from 700° C. to 750° C., from 700° C. to 725° C., from 725° C. to 850° C., from 725° C. to 825° C., from 725° C. to 800° C., from 725° C. to 775° C., from 725° C. to 750° C., from 750° C. to 850° C., from 750° C. to 825° C., from 750° C. to 800° C., from 750° C. to 775° C., from 775° C. to 850° C., from 775° C. to 825° C., from 775° C. to 800° C., from 800° C. to 850° C., from 800° C. to 825° C., from 825° C. to 850° C., or any combination of these ranges.

Referring still to FIG. 1, in non-limiting examples, the reactor system 103 described herein may be utilized to produce olefinic compounds from hydrocarbon feed streams. As used herein, the term “olefinic compounds” refers to hydrocarbons having one or more carbon-carbon double bonds apart from the formal double bonds in aromatic compounds. For example, ethylene and styrene are olefinic compounds, but ethylbenzene would not be an olefinic compound as the only double bonds present in ethylbenzene are formal double bonds present as part of the aromatic structure. Olefinic compounds may be produced from a variety of hydrocarbon feed streams by utilizing different reaction mechanisms. For example, olefinic compounds may be produced by at least dehydrogenation reactions, cracking reactions, dehydration reactions, and methanol-to-olefin reactions. These reaction types may utilize different feed streams and different particulate solids to produce olefinic compounds. It should be understood that when particulate solids are referred to herein, they may equally refer to the catalyst referenced with respect to the system of FIG. 1 and FIG. 2.

According to one or more embodiments, the reaction may be a dehydrogenation reaction. According to such embodiments, the one or more hydrocarbons may be a hydrocarbon feed stream. In one or more embodiments, the one or more hydrocarbons may comprise an alkyl moiety. As used in the present disclosure a hydrocarbon comprises an “alkyl moiety” if the molecule has at least one carbon-carbon single bond capable of being dehydrogenated to form a carbon-carbon double bond. The hydrocarbon feed stream may comprise one or more of ethylbenzene, ethane, propane, n-butane, and i-butane. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethylbenzene. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of ethylbenzene, ethane, propane, n-butane, and i-butane.

In one or more embodiments, the dehydrogenation reaction may utilize gallium and/or platinum particulate solids as a catalyst. In such embodiments, the particulate solids may comprise a gallium and/or platinum catalyst. As described herein, a gallium and/or platinum catalyst comprises gallium, platinum, or both. The gallium and/or platinum catalyst may be carried by an alumina or alumina silica support, and may optionally comprise potassium. Such gallium and/or platinum catalysts are disclosed in U.S. Pat. No. 8,669,406, which is incorporated herein by reference in its entirety. However, it should be understood that other suitable catalysts may be utilized to perform the dehydrogenation reaction.

In one or more embodiments, the reaction mechanism may be dehydrogenation followed by combustion (in the same chamber). In such embodiments, a dehydrogenation reaction may produce hydrogen as a byproduct, and an oxygen carrier material may contact the hydrogen and promote combustion of the hydrogen, forming water. Examples of such reaction mechanisms, which are contemplated as possible reactions mechanisms for the systems and methods described herein, are disclosed in WO 2020/046978 and U.S. Pat. Pub. No. 2021/0292259 the teachings of which are incorporated by reference in their entireties herein.

In one or more embodiments, the particulate solid may comprise an oxygen-carrier material and a dehydrogenation catalyst material. In some embodiments, the particulate solid may consist essentially of the oxygen-carrier material. As described herein, “consists essentially of” refers to materials with less than 1 wt. % of the non-recited materials (i.e., consisting essentially of A means A is at least 99 wt. % of the composition). In some embodiments, the particulate solid may not comprise a dehydrogenation catalyst material. In some embodiments, the oxygen-carrier material and the dehydrogenation catalyst material may be separate particles of the particulate solid. In some embodiments, the oxygen-carrier material and the dehydrogenation catalyst may be contained in the same particles of the particulate solid.

In embodiments where the particulate solid comprises a dehydrogenation catalyst, the dehydrogenation of the one or more hydrocarbons may be at least partially by catalytic dehydrogenation. Catalytic dehydrogenation is the dehydrogenation of a hydrocarbon that is promoted by the use of a dehydrogenation catalyst. In embodiments, where the particulate solid does not comprise a dehydrogenation catalyst the dehydrogenation reaction may be a non-catalytic thermal dehydrogenation reaction. Non-catalytic thermal dehydrogenation refers to the dehydrogenation of a hydrocarbon that occurs without the use of a dehydrogenation catalyst and instead may occur because of high temperature, pressure or combinations thereof.

In some embodiments, the particulate solid may comprise a “dual-purpose material” that may act as both a dehydrogenation catalyst as well as an oxygen-carrier material. It should be understood that, in at least the embodiments described herein where an oxygen-carrier material and a dehydrogenation catalyst are utilized in the same reaction vessel (such as those of FIG. 1), such a dual-purpose material may be utilized either in replacement or in combination with the oxygen-carrier material of the particulate solid or the dehydrogenation catalyst of the particulate solid.

In one or more embodiments, the particulate solid may be capable of fluidization. In some embodiments, the particulate solid may exhibit properties known in the industry as “Geldart A” or “Geldart B” properties. Particles may be classified as “Group A” or “Group B” according to D. Geldart, Gas Fluidization Technology, John Wiley & Sons (New York, 1986), 34-37; and D. Geldart, “Types of Gas Fluidization,” Powder Technol. 7 (1973) 285-292, which are incorporated herein by reference in their entireties.

Group A is understood by those skilled in the art as representing an aeratable powder, having a bubble-free range of fluidization; a high bed expansion; a slow and linear deaeration rate; bubble properties that may include a predominance of splitting/recoalescing bubbles, with a maximum bubble size and large wake; high levels of solids mixing and gas backmixing, assuming equal U-Umf (U is the velocity of the carrier gas, and Umf is the minimum fluidization velocity, typically though not necessarily measured in meters per second, m/s, i.e., there is excess gas velocity); axisymmetric slug properties; and no spouting, except in very shallow beds. The properties listed tend to improve as the mean particle size decreases, assuming equal cfp; or as the <45 micrometers (μm) proportion is increased; or as pressure, temperature, viscosity, and density of the gas increase. In general, the particles may exhibit a small mean particle size and/or low particle density (<1.4 grams per cubic centimeter, g/cm³), fluidize easily, with smooth fluidization at low gas velocities, and may exhibit controlled bubbling with small bubbles at higher gas velocities.

Group B is understood by those skilled in the art as representing a “sand-like” powder that starts bubbling at Umf; that exhibits moderate bed expansion; a fast deaeration; no limits on bubble size; moderate levels of solids mixing and gas backmixing, assuming equal U-Umf; both axisymmetric and asymmetric slugs; and spouting in only shallow beds. These properties tend to improve as mean particle size decreases, but particle size distribution and, with some uncertainty, pressure, temperature, viscosity, or density of gas seem to do little to improve them. In general, most of the particles having a particle size (cfp) of 40 μm<cfp<500 μm when the density (pp) is 1.4<pp<4 g/cm³, and preferably 60 μm<cfp<500 μm when the density (pp) is 4 g/cm³ and 250 μm<cfp<100 μm when the density (pp) is 1 g/cm³.

In one or more embodiments, the olefinic compounds may be present in a “product stream” sometimes called an “olefin-containing effluent”. Such a stream exits the reactor system of FIG. 1 and may be subsequently processed. In one or more embodiments, the olefinic compounds may comprise one or more of ethylene, propylene, butylene, or styrene. The term butylene includes any isomers of butylene, such as α-butylene, cis-β-butylene, trans-β-butylene, and isobutylene. In some embodiments, the olefin-containing effluent may comprise at least 20 wt. %, at least 30 wt. %, at least 40 wt. %, at least 50 wt. %, or even at least 60 wt. % of ethylene. In additional embodiments, the olefin-containing effluent may comprise at least 20 wt. %, at least 30 wt. %, at least 40 wt. %, at least 50 wt. %, or even at least 60 wt. % of propylene. In additional embodiments, the olefin-containing effluent may comprise at least 20 wt. %, at least 30 wt. %, at least 40 wt. %, at least 50 wt. %, or even at least 60 wt. % of butylene. In additional embodiments, the olefin-containing effluent may comprise at least 20 wt. %, at least 30 wt. %, at least 40 wt. %, at least 50 wt. %, or even at least 60 wt. % of styrene. In additional embodiments, the olefin-containing effluent may comprise at least 20 wt. %, at least 30 wt. %, at least 40 wt. %, at least 50 wt. %, or even at least 60 wt. % of the sum of one or more of ethylene, propylene, butylene, and styrene. The olefin-containing effluent may further comprise unreacted components of the feed stream, as well as other reaction products that are not considered light olefins. The olefinic compounds may be separated from unreacted components in subsequent separation steps.

### EXAMPLES

### Example 1—Catalyst Deactivation from CH₄ and Steam

In Example 1, the effect of CH₄ exposure and steam exposure on catalyst activity was observed. The testing was carried out in a fixed-bed rig using 0.5 g of a supported gallium catalyst loaded with a platinum promoter. Lab simulated reaction-combustion-reactivation cycles were run in the fixed-bed rig. In each cycle a dehydrogenation reaction was first conducted at 625° C. with weight hourly space velocity “WHSV” propane of 10 hr⁻¹ and feed composition of 90% propane/10% nitrogen for 60 seconds, the catalyst was then treated with a simulated combustion stream at 750° C. for 3 minutes, finally the catalyst was reactivated under air at 750° C. for 15 minutes. Dehydrogenation performance was collected at 15 seconds time on stream. To test the effect of CH₄ exposure on catalyst activity the catalyst was treated with 100% CH₄ with a flow rate of 10 standard cubic centimeters per minute (sccm) at 750° C. for 2 minutes after reactivation under air. The cycle was then run again and the dehydrogenation performance of the CH₄ treated catalyst was collected at 15 seconds time on stream. Ten lab simulated reaction-combustion-reactivation cycles were then run without CH₄ treatment to restore catalyst activity to baseline. The catalyst was treated with 100% CH₄ at a lower temperature, 625° C. for 2 minutes. The cycle was then run again and the dehydrogenation performance of the low temperature CH₄ treated catalyst was collected at 15 seconds time on stream.

Ten lab simulated reaction-combustion-reactivation cycles were then run without CH₄ treatment to restore catalyst activity to baseline. The catalyst was then treated with steam with a flow rate of 24 sccm at 625° C. for 2 mins, followed by a 5 minute stripping under helium before the catalyst was run in a lab simulated reaction-combustion reactivation cycle. Dehydrogenation performance of the steam treated catalyst was collected at 15 seconds time on stream. The dehydrogenation performance of the catalyst under the various treatment conditions was recorded in Table 1.

TABLE 1

| | | Propane | Propylene | Intrinsic Rate [mol |
|---|---|---|---|---|
| | | Conversion | Selectivity | propane/hr/gm |
| Sample | Treatment | [%] | [%] | cat/psi] |
| A | n/a | 46.1 | 96.4 | 0.0422 |
| B1 | CH₄ (750° C., | 29.3 | 94.0 | 0.0137 |
| | 2 min) | | | |
| B2 | CH₄ (625° C., | 45.8 | 95.7 | 0.0413 |
| | 2 min) | | | |
| C | Steam | 18.2 | 89.2 | 0.0058 |

As indicated by Table 1, exposing the catalyst to steam or CH₄ at high temperature (750° C.) prior to using the catalyst in a dehydrogenation reaction negatively affected the propane conversion and the propane selectivity performance of the catalyst as well as the intrinsic rate of the catalyst. As seen with Sample B1, exposure to CH₄ caused approximately 40% loss in propane conversion performance, a 2.4% loss in propane selectivity, and a 68% loss in activity when compared to a catalyst that had not been exposed to CH₄ prior to use in a dehydrogenation reaction (i.e. Sample A). Similarly, Sample C, shows that catalyst exposure to steam prior to use in a dehydrogenation reaction caused an approximately 60% loss in propane conversion performance, an 8% loss in propane selectivity, and a 86% loss in activity when compared to a catalyst that had not been exposed to steam prior to use in a dehydrogenation reaction (i.e. Sample A). This demonstrates that premixing of high temperature regenerated catalyst which carries oxygen with the low temperature recycled deactivated catalyst which carries stripping hydrocarbon gases such as methane can cause unexpected deactivation of the regenerated catalyst. In contrary, exposing the catalyst to CH₄ at low temperature (625° C.) prior to using the catalyst in a dehydrogenation reaction only has marginal effect on the propane conversion and the propane selectivity performance of the catalyst as well as the intrinsic rate of the catalyst.

### Example 2—Effect of Catalyst Distributor Configuration

To simulate the catalyst mixing and dehydrogenation reactions within a typical fluidized catalytic dehydrogenation reactor, a reactor model consisting of three continuously stirred tank reactors (CSTR) in series was utilized as shown in FIG. 3, where in each CSTR, catalyst and reactants are well mixed. In FIG. 3, the three CSTRs are shown as part of a single reactor system 600. Catalyst and/or reactants flow from the first CSTR 610 to the second CSTR 620 to the third CSTR 630. For the case where there is premixing of regenerated catalyst 640 and 1^{st} portion of deactivated catalyst 606 upstream of the reactor, it was represented by a mixer (mix pot) 640. The catalytic dehydrogenation and thermal reactions were solved using kinetics models by Lobera et. al. (2008) and Sundaram & Froment (1977). Mass and energy conservation equations were solved for the mixer and each CSTR to determine the effluent gas composition and temperature. Fifty percent of the catalyst from the third CSTR 630 was recycled as recycled catalyst 606 for use again in the reactor simulation without undergoing regeneration. The catalyst will continuously deactivate as it moves from CSTR to CSTR due to the net amount of propane reacted in each CSTR. The same total catalyst amount in each CSTR was used in simulating each reactor configuration shown below. The reactor model utilized the same regeneration process for each configuration resulting in the same catalyst activity for the regenerated catalyst 604 with adsorbed oxygen. The regenerated catalyst was at the temperature of 750° C. The model also utilized CH₄ as a stripping gas in all configurations, which resulted in the recycled catalyst adsorbing some of the CH₄. The recycled catalyst temperature was predicted as the effluent temperature of the third CSTR.

Three reactor configurations were evaluated using the reactor model described above. In Configuration A, as shown in FIG. 3A, regenerated catalyst 604 carrying adsorbed oxygen is mixed with recycled catalyst 606 from the third CSTR 630 in a mix pot 640 upstream of the first CSTR 610. The mixed catalyst 642 then enters the first CSTR 610, where the propane 602 is fed into the reactor 600. Based on the experimental data presented in Example 1, it was estimated that the adsorbed CH₄ in the recycled catalyst 606 reacts with residual oxygen in the regenerated catalyst 604 at a mix pot 640 temperature of 685° C., resulting in 40% activity. Configuration A, approximates the reactor conditions that would occur if the recycled catalyst 606 and the regenerated catalyst 604 are premixed in a mix pot 640 before entering the reactor 600.

In Configuration B, as shown in FIG. 3B, recycled catalyst 606 from the third CSTR 630 is fed into the first CSTR 610, propane 602 is also fed separately into the first CSTR 610, and the regenerated catalyst 604 is fed into the second CSTR 620. Accordingly, the recycled catalyst 606 passes from the third CSTR 630 to the first CSTR 610 and then passes to the second CSTR 620 before mixing with the regenerated catalyst 604 that is fed to the second CSTR 620. Due to the endothermic dehydrogenation reaction, the process temperature decreases following the CSTR's in series. The mixing of the regenerated catalyst 604 carrying adsorbed oxygen and recycled catalyst 606 with adsorbed CH₄ took place in the second CSTR 620, where the temperature was predicted to be 623.5° C. As shown in Example 1, the impact of CH₄ on catalyst activity is significantly reduced at lower temperatures and was subsequently neglected for configuration B.

In Configuration 1, as shown in FIG. 3C, regenerated catalyst 604 is fed into the first CSTR 610, propane 602 is also fed separately into the first CSTR 610, and the recycled catalyst 606 from the third CSTR 630 is separately fed into the second CSTR 620. Accordingly, the regenerated catalyst 604 passes from the first CSTR 610 into the second CSTR 620 before mixing with the recycled catalyst 606 in the second CSTR 620. The adsorbed oxygen in the regenerated catalyst 604 was estimated to be consumed in the first CSTR 610 before mixing with the recycled catalyst 606 with adsorbed CH₄. Therefore, the deactivation of CH₄ in catalyst dehydrogenation activity was neglected. Configuration 1 represents the reactor conditions that would occur if the regenerated catalyst 604 was fed into the reactor 600 in a position between the propane feed 602 and the recycled catalyst 604. The results from the runs of Configurations A, B, and 1 were recorded in Table 2.

TABLE 2

| | Temperature in | Temperature in | Overall | | |
|---|---|---|---|---|---|
| | mixer upstream | CSTR's 1/2/3 | Propane | Propylene | Propylene |
| Configuration | of reactor [° C.] | [° C.] | Conversion | Selectivity | Yield |
| A | 685 | 647.9/631.1/611.6 | 27.2% | 87.2% | 23.7% |
| B | N/A | 574.3/631.8/613.1 | 33.9% | 92.6% | 31.4% |
| 1 | N/A | 653.3/625.6/611.9 | 36.3% | 90.2% | 32.7% |

As indicated by Table 2, Configuration 1 based on the present invention has the highest overall propane conversion, at 36.3% and yield of propylene, at 32.7%, of the three configurations. The next closest propane conversion is Configuration B, which has a propane conversion and propylene yield 2.4% and 1.3% less than Configuration 1 respectively, showing that Configuration 1 has significantly better propane conversion compared to the other tested configurations.

In a first aspect of the present disclosure, one or more olefinic compounds may be produced by a method comprising dehydrogenating a feed stream in the presence of a catalyst in a reactor to form a product stream and a deactivated catalyst. The feed stream comprises one or more hydrocarbons comprising an alkyl moiety and the product stream comprises one or more olefinic compounds. The method also comprises separating the deactivated catalyst into a first portion of deactivated catalyst and a second portion of deactivated catalyst. The method also comprises passing the second portion of deactivated catalyst to a regenerator and processing the second portion of deactivated catalyst in the regenerator to form a regenerated catalyst. The method also comprises passing the first portion of deactivated catalyst and the regenerated catalyst to the reactor. The first portion of deactivated catalyst enters the reactor downstream of the regenerated catalyst relative to the flow direction of the feed stream. The first portion of deactivated catalyst has a lower temperature than the regenerated catalyst.

A second aspect of the present disclosure includes any previous aspect or combination of aspects, where the first portion of deactivated catalyst and the regenerated catalyst are passed to the reactor through a particulate solids distributor that separately passes the first portion of deactivated catalyst and the regenerated catalyst into the reactor.

A third aspect of the present disclosure includes any previous aspect or combination of aspects, where the particulate solids distributor extends into the reactor through a bottom end of the reactor.

A fourth aspect of the present disclosure includes any previous aspect or combination of aspects, where the one or more hydrocarbons comprise propane and the one or more olefinic compounds comprise propylene.

A fifth aspect of the present disclosure includes any previous aspect or combination of aspects, where the catalyst comprises one or more of gallium or platinum.

A sixth aspect of the present disclosure includes any previous aspect or combination of aspects, where the reactor operates as a fast fluidized, turbulent, or bubbling fluidized bed reactor.

A seventh aspect of the present disclosure includes any previous aspect or combination of aspects, where a temperature of the first portion of deactivated catalyst passed to the reactor is from 580° C. to 800° C.

An eighth aspect of the present disclosure includes any previous aspect or combination of aspects, where a temperature of the regenerated catalyst passed to the reactor is from 680° C. to 900° C.

A ninth aspect of the present disclosure includes any previous aspect or combination of aspects, where hydrocarbons are entrained in the first portion of deactivated catalyst and the second portion of deactivated catalyst.

A tenth aspect of the present disclosure includes any previous aspect or combination of aspects, where the one or more hydrocarbons comprise propane and the one or more olefinic compounds comprise propylene. The catalyst comprises one or more of gallium and platinum. The temperature of the first portion of deactivated catalyst passed to the reactor is from 580° C. to 800° C. The temperature of the regenerated catalyst passed to the reactor is from 680° C. to 900°. The first portion of deactivated catalyst and the regenerated catalyst are passed to the reactor through a particulate solids distributor that separately passes the first portion of deactivated catalyst and the regenerated catalyst into the reactor. The particulate solids distributor extends into the reactor through the bottom of the reactor. The reactor operates as a fast fluidized, turbulent, or bubbling fluidized bed reactor.

An eleventh aspect of the present disclosure includes any previous aspect or combination of aspects, where the reactor comprises a feed distribution plate, the regenerated catalyst enters the reactor between the feed distribution plate and the first portion of deactivated catalyst and the temperature of the feed distribution plate is from 25° C. to 700° C.

A twelfth aspect of the present disclosure includes any previous aspect or combination of aspects, where the first portion of deactivated catalyst and the regenerated catalyst make up at least 95 wt. % of the catalyst passed to the reactor.

A thirteenth aspect of the present disclosure includes any previous aspect or combination of aspects, where the mass flow rate ratio of the first portion of deactivated catalyst to regenerated catalyst is from 0.1 to 5.

A fourteenth aspect of the present disclosure includes any previous aspect or combination of aspects, where the deactivated catalyst is separated into a third portion in addition to the first portion of deactivated catalyst and the second portion of deactivated catalyst. The third portion of deactivated catalyst is combined with the regenerated catalyst prior to being passed to the reactor to form a mixed catalyst.

A fifteenth aspect of the present disclosure includes any previous aspect or combination of aspects, where the mixed catalyst has a temperature of from 600° C. to 850° C.

It will be apparent to those skilled in the art that various modifications and variations can be made to the presently disclosed technology without departing from the spirit and scope of the technology. Since modifications combinations, sub-combinations and variations of the disclosed embodiments incorporating the spirit and substance of the presently disclosed technology may occur to persons skilled in the art, the technology should be construed to include everything within the scope of the appended claims and their equivalents. Additionally, although some aspects of the present disclosure may be identified herein as preferred or particularly advantageous, it is contemplated that the present disclosure is not limited to these aspects.

It is noted that the various details described in this disclosure should not be taken to imply that these details relate to elements that are essential components of the various embodiments described in this disclosure, even in cases where a particular element is illustrated in each of the drawings that accompany the present description. Unless specifically identified as such, no feature disclosed and described herein should be construed as “essential”. Contemplated embodiments of the present technology include those that include some or all of the features of the appended claims.

For the purposes of describing and defining the present disclosure it is noted that the term “about” are utilized in this disclosure to represent the inherent degree of uncertainty that may be attributed to any quantitative comparison, value, measurement, or other representation. The term “about” are also utilized in this disclosure to represent the degree by which a quantitative representation may vary from a stated reference without resulting in a change in the basic function of the subject matter at issue.

In relevant cases, where a composition is described as “comprising” one or more elements, embodiments of that composition “consisting of” or “consisting essentially of” those one or more elements is contemplated herein.

It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

It is noted that one or more of the following claims and the detailed description utilize the terms “where” or “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that any two quantitative values assigned to a property may constitute a range of that property, and all combinations of ranges formed from all stated quantitative values of a given property are contemplated in this disclosure. Where multiple ranges for a quantitative value are provided, these ranges may be combined to form a broader range, which is contemplated in the embodiments described herein.

As would be understood in the context of the term as used herein, the term “passing” may include directly passing a substance between two portions of the disclosed system and, in some other instances, to mean indirectly passing a substance between two portions of the disclosed system. For example, indirect passing may include steps where the named substance passes through an intermediate operations unit, valve, sensor, etc.

1. A method for producing one or more olefinic compounds, the method comprising:
dehydrogenating a feed stream in the presence of a catalyst in a reactor to form a product stream and a deactivated catalyst, wherein the feed stream comprises one or more hydrocarbons comprising an alkyl moiety and the product stream comprises one or more olefinic compounds;
separating the deactivated catalyst into a first portion of deactivated catalyst and a second portion of deactivated catalyst;
passing the second portion of deactivated catalyst to a regenerator;
processing the second portion of deactivated catalyst in the regenerator to form a regenerated catalyst; and
passing the first portion of deactivated catalyst and the regenerated catalyst to the reactor, wherein the first portion of deactivated catalyst enters the reactor downstream of the regenerated catalyst relative to the flow direction of the feed stream, and wherein the first portion of deactivated catalyst has a lower temperature than the regenerated catalyst.

2. The method of claim 1, wherein the first portion of deactivated catalyst and the regenerated catalyst are passed to the reactor through a particulate solids distributor that separately passes the first portion of deactivated catalyst and the regenerated catalyst into the reactor.

3. The method of claim 2, wherein the particulate solids distributor extends into the reactor through a bottom end of the reactor.

4. The method of claim 1, wherein the one or more hydrocarbons comprise propane and the one or more olefinic compounds comprise propylene.

5. The method of claim 1, wherein the catalyst comprises one or more of gallium and platinum.

6. The method of claim 1, wherein the reactor operates as a fast fluidized, turbulent, or bubbling fluidized bed reactor.

7. The method of claim 1, wherein a temperature of the first portion of deactivated catalyst passed to the reactor is from 580° C. to 800° C.

8. The method of claim 1, wherein a temperature of the regenerated catalyst passed to the reactor is from 680° C. to 900° C.

9. The method of claim 1, wherein hydrocarbons are entrained in the first portion of deactivated catalyst and the second portion of deactivated catalyst.

10. The method of claim 1, wherein:
the one or more hydrocarbons comprise propane and the one or more olefinic compounds comprise propylene;
the catalyst comprises one or more of gallium and platinum;
the temperature of the first portion of deactivated catalyst passed to the reactor is from 580° C. to 800° C.;
the temperature of the regenerated catalyst passed to the reactor is from 680° C. to 900° C.; and
wherein the first portion of deactivated catalyst and the regenerated catalyst are passed to the reactor through a particulate solids distributor that separately passes the first portion of deactivated catalyst and the regenerated catalyst into the reactor;
the particulate solids distributor extends into the reactor through the bottom of the reactor; and
the reactor operates as a fast fluidized, turbulent, or bubbling fluidized bed reactor.

11. The method of claim 1, wherein the reactor comprises a feed distribution plate, the regenerated catalyst enters the reactor between the feed distribution plate and the first portion of deactivated catalyst, and the temperature of the feed distribution plate is from 25° C. to 700° C.

12. The method of claim 1, wherein the first portion of deactivated catalyst and the regenerated catalyst make up at least 95 wt. % of the catalyst passed to the reactor.

13. The method of claim 1, wherein the mass flow rate ratio of the first portion of deactivated catalyst to the regenerated catalyst is from 0.1 to 5.

14. The method of claim 1, wherein the deactivated catalyst is separated into a third portion in addition to the first portion of deactivated catalyst and the second portion of deactivated catalyst, and wherein the third portion of deactivated catalyst is combined with the regenerated catalyst prior to being passed to the reactor to form a mixed catalyst.

15. The method of claim 14, wherein the mixed catalyst has a temperature of from 600° C. to 850° C.
